(19) United States
(12) Patent Application Publication
(10) Pub. No.: US 2007/0167495 A1
(43) Pub. Date: Jul. 19, 2007

(54) Pleuromutilin derivatives, process for their preparation and uses thereof

(75) Inventors: Pamela Brown, Essex (GB); Eric Hunt, Essex (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: GLAXOSMITHKLINE SERVICES UNLIMITED, Middlesex (GB)

(21) Appl. No.: 10/552,238

(22) PCT Filed: Apr. 6, 2004
(86) PCT No.: PCT/EP04/03783

§ 371 Date: Jan. 3, 2007

(30) Foreign Application Priority Data
Apr. 8, 2003 (GB) 0308114.8

(51) Int. Cl. A61K 31/4412; A61K 31/22; C07C 323/06

(52) U.S. Cl. 514/345; 514/550; 546/285; 560/147

(57) ABSTRACT

Pleuromutilin compounds of the formula: 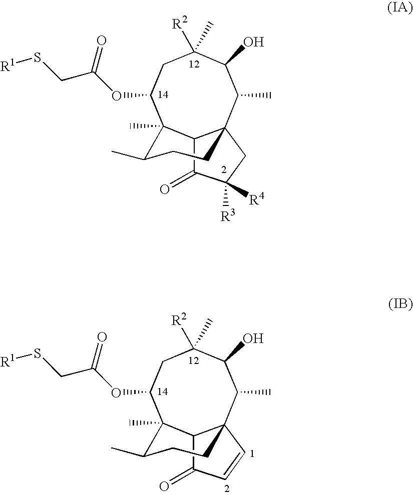 are of use in antibacterial therapy.

The present invention relates to novel compounds, to processes for their preparation, to pharmaceutical compositions containing them and to their use in medical therapy, particularly antibacterial therapy.

Pleuromutilin, the compound of formula (A), is a naturally occurring antibiotic which has antimycoplasmal activity and modest antibacterial activity. Mutilin and other compounds with a free OH at C-14 are inactive. The impact of further modification at C-14 on the activity of pleuromutilin has been investigated (H. Egger and H. Reinshagen, J. Antibiotics, 1976, 29, 923). Replacing the hydroxy group of the glycolic ester moiety at position 14 by another O, S or N-linked group was found to improve anti-microbial activity. Egger and Reinshagen also described the preparation of (2-carboxylato-phenylsulfanyl)-acetic acid mutilin 14-ester. However, they found that this derivative was inferior in activity even to the parent substance pleuromutilin. Introducing a diethylaminoethylthio group gives the compound of formula (B), also known as Tiamulin, which is used as a veterinary antibiotic (G. Hogenauer in Antibiotics, Vol. V, part 1, ed. F. E. Hahn, Springer-Verlag, 1979, p. 344).

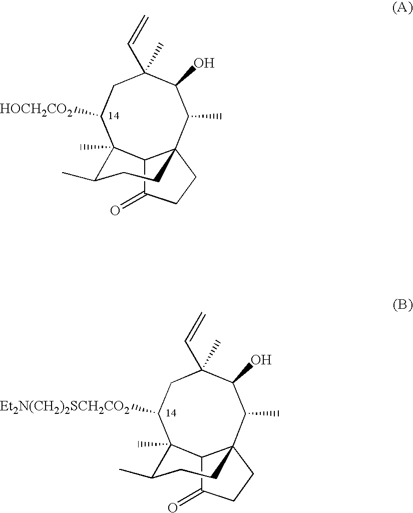

In this application, the non-conventional numbering system which is generally used in the literature (G. Hogenauer, loc. cit.) is used.

WO 97/25309 (SmithKline Beecham) describes further modification of the acyloxy group, disclosing 14-O-carbamoyl derivatives of mutilin or 19,20-dihydromutilin, in which the N-atom of the carbamoyl group is unsubstituted, mono- or di-substituted.

WO98/05659 (SmithKline Beecham) discloses 14-O-carbamoyl derivatives of mutilin or 19,20-dihydromutilin, in which the N-atom of the carbamoyl group is acylated by a group which includes an azabicyclic moiety.

WO 99/21855 (SmithKline Beecham) describes further derivatives of mutilin or 19,2-dihydromutilin, in which the glycolic ester moiety at position 14 is replaced by the group R²(CH₂)ₘX(CH₂)ₙCH₂COO— in which R² is a non-aromatic mono- or bicyclic group.

WO 00/27790 (SmithKline Beecham) describes C-14 spirocyclic, acylcarbamate, heteroaryalkyl carboxylate or arylalkoxyalkyl carboxylate derivatives of mutilin or 19,20-dihydromutilin.

WO 00/37074 (SmithKline Beecham) describes further derivatives of mutilin or 19,20-dihydromutilin having a heteroaryl acetate substituent at the C-14 position.

WO 00/73287 (SmithKline Beecham) describes further derivatives of mutilin or 19,20-dihydromutilin having an isoxazoline carboxylate substituent at the C-14 position.

WO 01/14310 (SmithKline Beecham) describes further derivatives of mutilin or 19,20-dihydromutilin having a β-ketoester substituent at the C-14 position.

WO 01/74788 (SmithKline Beecham) describes 2-hydroxymutilin carbamate derivatives.

WO 02/12199 (SmithKline Beecham) describes derivatives having a heterocyclic ester substituent at the C-14 position.

WO 02/30929 (SmithKline Beecham) describes derivatives having an oxycarbonyl carbamate substituent at the C-14 position.

WO 02/38528 (SmithKline Beecham) describes derivatives having a malonamide or malonic ester substituent at the C-14 position.

In addition, 19,20-dihydro-2α-hydroxy-mutilin is described by G. Schulz and H. Berner in Tetrahedron, 1984, vol. 40, pp 905-917, and a number of C-14 ether, carbamate, amide and urea derivatives of mutilin or 19,20-dihydromutilin are described by Brooks et al. in Bioorg. Med. Chem, 2001, vol. 9, pp 1221-1231.

The present invention is based on the unexpected discovery that novel mutilin derivatives having an aromatic carboxylic acid substituent at the 14-position also have potent antimicrobial activity.

Accordingly, the present invention provides a compound of formula (IA) or (IB): 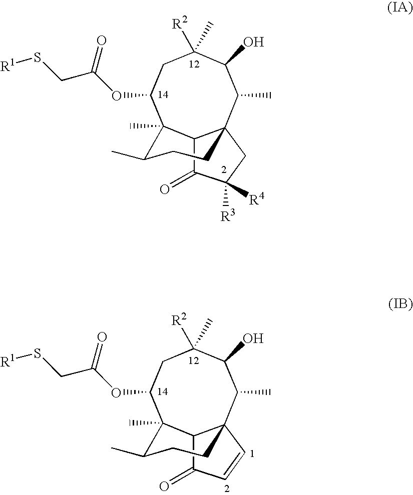
in which:

R¹ is a five- or six-membered aryl or heteroaryl ring substituted by a carboxylic acid group and optionally further substituted by up to four groups independently selected from halogen, (C₁₋₆)alkyl, aryl, aryl(C₁₋₆)alkyl, (C₁₋₆)alkoxy, (C₁₋₆)alkoxy(C₁₋₆)alkyl, halo(C₁₋₆)alkyl, aryl(C₁₋₆)alkoxy, hydroxy, nitro, cyano, azido, amino, mono- and di-N-(C₁₋₆)alkylamino, acylamino, arylcarbonylamino, acyloxy, carbamoyl, mono- and di-N-(C₁₋₆)alkylcarbamoyl, (C₁₋₆)alkoxycarbonyl, aryloxycarbonyl, ureido, guanidino, (C₁₋₆)alkylguanidino, amidino, (C₁₋₆)alkylamidino, sulphonylamino, aminosulphonyl, (C₁₋₆)alkylthio, (C₁₋₆)alkylsulphinyl, (C₁₋₆)alkylsulphonyl, heterocyclyl, heteroaryl, heterocyclyl(c₁₋₆)alkyl and heteroaryl(C₁₋₆)alkyl, or two adjacent ring carbon atoms may be linked by a (C₃₋₅)alkylene chain, to form a carbocyclic ring;

R² is vinyl or ethyl; and

R³ is hydrogen, hydroxy or fluorine and R⁴ is hydrogen,

or R³ is hydrogen and R⁴ is fluorine;

or a pharmaceutically acceptable derivative thereof;

with the proviso that the compound of formula (IA) is not (2-carboxylato-phenylsulfanyl)-acetic acid mutilin 14-ester.

In one embodiment of the present invention, there is provided a compound of formula (IA).

In one embodiment of the present invention, R¹ is a five- or six-membered aryl ring or a five- or six-membered heteroaryl ring containing up to three, preferably one or two, heteroatoms independently selected from nitrogen, sulphur or oxygen, for example phenyl, furyl, thiophenyl, pyridyl, imidazolyl, 1,2,4-triazolyl, thiazolyl, pyrazolyl, pyridazinyl, pyrimidinyl, pyrazinyl or 1,2,4-triazinyl, substituted by a carboxylic acid group. In another embodiment, R¹ is a six-membered aryl ring or a six-membered heteroaryl ring containing one or two nitrogen atoms, for example phenyl or pyridyl, substituted by a carboxylic acid group.

The carboxylic acid group may be attached at any position on the aryl or heteroaryl ring. Preferably the carboxylic acid group is attached at a position which is not adjacent to the point of attachment of the sulphur position, for example when R¹ is a six-membered aryl ring the carboxylic acid group is preferably meta or para to the point of attachment of the sulphur atom.

In one embodiment of the present invention, R¹ may be optionally further substituted by up to four groups, for example one or two groups, independently selected from halogen, (C₁₋₆)alkyl, (C₁₋₆)alkoxy, hydroxy, cyano, amino, and mono- and di-N-(C₁₋₆)alkylamino.

In one embodiment of the present invention, R² is vinyl.

In one embodiment of the present invention, R³ and R⁴ are H.

When used herein, the term “aryl” refers to, unless otherwise defined, single or fused aromatic rings suitably containing from 4 to 7, preferably 5 or 6, ring atoms in each ring. A fused ring system may include aliphatic rings and need only include one aromatic ring. Examples of suitable aryl rings include phenyl and naphthyl.

When used herein, the term “heteroaryl” suitably includes, unless otherwise defined, a mono- or bicyclic heteroaromatic ring system comprising up to four, preferably one or two, heteroatoms each selected from nitrogen, sulphur and oxygen. Each ring may have from 4 to 7, preferably 5 or 6, ring atoms. A bicyclic heteroaromatic ring system may include a carbocyclic ring. Examples of suitable heteroaryl rings include furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, triazolyl, Tetrazolyl and quinazolinyl.

When used herein, the terms “halogen” or “halo” refer to a fluorine, chlorine, bromine or iodine atom.

When used herein, the term “(C₁₋₆)alkyl” refers to (individually or as part of another group) straight and branched groups containing up to six carbon atoms. Examples of such groups include methyl, ethyl, propyl, isopropyl, n-butyl, isobutyl, tert-butyl, pentyl or hexyl.

When used herein, the term “(C₁₋₆)alkoxy” refers to straight and branched chain alkoxy groups, for example methoxy, ethoxy, propoxy, prop-2-oxy, butoxy, but-2-oxy or methylprop-2-oxy.

When used herein, the term “halo(C₁₋₆)alkyl” refers to an alkyl group having up to six carbon atoms and wherein at least one hydrogen atom is replaced with halogen such as, for example, a trifluoromethyl group.

When used herein, the term “acyl” refers to (C₁₋₆)alkylcarbonyl, for example formyl.

When used herein, the terms “heterocyclyl” and “heterocyclic” refer to non-aromatic, single and fused, rings suitably containing up to four heteroatoms in each ring, each of which is independently selected from oxygen, nitrogen and sulphur, and wherein the nitrogen and sulphur heteroatoms may be optionally oxidised, and the nitrogen atom may be optionally quarternized. Each heterocyclic ring preferably has from 4 to 7, preferably 5 or 6, ring atoms. A fused heterocyclic ring system may include carbocyclic rings and need include only one heterocyclic ring. The heterocyclic ring may be attached via any heteroatom or carbon atom. Examples of suitable heterocylic rings include morpholinyl, pyrrolidinonyl, pyrrolidinyl, piperidinyl, hydantoinyl, valerolactamyl, oxiranyl, oxetanyl, Tetrahydrofuranyl, Tetrahydropyranyl, Tetrahydropyridinyl, Tetrahydropyrimidinyl, Tetrahydrothiophenyl, Tetrahydrothiopyranyl, Tetrahydropyrimidinyl, Tetrahydrothiophenyl and Tetrahydrothiopyranyl.

When used herein, the term “alkylene” refers to straight and branched chain saturated hydrocarbon linker groups. Examples of alkylene groups include methylene (—CH₂—) and ethylene (—CH₂CH₂—).

With regard to stereoisomers, depending on the substituents, two or more diastereomers of the compounds of formula (IA) and (IB) may be possible. The present invention includes all such individual diastereomers and mixtures thereof.

The 2-hydroxy-substituted compounds of formula (IA) are of the (2S) configuration. The 2-F-substituted compounds of formula (IA) may of (2S) configuration or (2R) configuration, or be provided as mixtures thereof. The (2S) configuration is however preferred.

Representative compounds of the invention include:
(4-carboxylato-phenylsulfanyl)-acetic acid mutilin 14-ester;
(4-carboxylato-phenylsulfanyl)-acetic acid 19,20-dihydro-mutilin 14-ester;
(3-carboxylato-phenylsulfanyl)-acetic acid mutilin 14-ester; and
(5-carboxylato-pyridin-2-yl-sulfanyl)-acetic acid mutilin 14-ester;
   and pharmaceutically acceptable derivatives thereof.

When used herein, the term “pharmaceutically acceptable” means a compound which is suitable for pharmaceutical use. Salts and solvates of compounds of the invention which are suitable for use in medicine are those wherein the counterion or associated solvent is pharmaceutically acceptable. However, salts and solvates having non-pharmaceutically acceptable counterions or associated solvents are within the scope of the present invention, for example, for use as intermediates in the preparation of other compounds of the invention and their pharmaceutically acceptable salts and solvates.

When used herein, the term “pharmaceutically acceptable derivative”, means any pharmaceutically acceptable salt, solvate, or prodrug, e.g. ester, of a compound of the invention, which upon administration to the recipient is capable of providing (directly or indirectly) a compound of the invention, or an active metabolite or residue thereof. Such derivatives are recognizable to those skilled in the art, without undue experimentation. Nevertheless, reference is made to the teaching of Burger's Medicinal Chemistry and Drug Discovery, 5^{th} Edition, Vol 1: Principles and Practice, which is incorporated herein by reference to the extent of teaching such derivatives. Preferred pharmaceutically acceptable derivatives are salts, solvates, esters, carbamates and phosphate esters. Particularly preferred pharmaceutically acceptable derivatives are salts, solvates and esters. Most preferred pharmaceutically acceptable derivatives are salts and esters, in particular salts.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compound of the invention are within the scope of the invention. This invention includes within its scope stoichiometric hydrates as well as compounds containing variable amounts of water.

As used herein, the term “prodrug” means a compound which is converted within the body, e.g. by hydrolysis in the blood, into its active form that has medical effects. Pharmaceutically acceptable prodrugs are described in T. Higuchi and V. Stella, Prodrugs as Novel Delivery Systems, Vol. 14 of the A.C.S. Symposium Series; Edward B. Roche, ed., Bioreversible Carriers in Drug Design, American Pharmaceutical Association and Pergamon Press, 1987; and in D. Fleisher, S. Ramon and H. Barbra “Improved oral drug delivery: solubility limitations overcome by the use of prodrugs”, Advanced Drug Delivery Reviews (1996) 19(2) 115-130, each of which are incorporated herein by reference.

Prodrugs are any covalently bonded carriers that release a compound of structure (I) in vivo when such prodrug is administered to a patient. Prodrugs are generally prepared by modifying functional groups in a way such that the modification is cleaved, either by routine manipulation or in vivo, yielding the parent compound. Prodrugs include, for example, compounds of this invention wherein hydroxy or amine groups are bonded to any group that, when administered to a patient, cleaves to form the hydroxy or amine groups. Thus, representative examples of prodrugs include (but are not limited to) acetate, formate and benzoate derivatives of alcohol and amine functional groups of the compounds of structure (I). Further, in the case of a carboxylic acid (—COOH), esters may be employed, such as methyl esters, ethyl esters, and the like. Esters may be active in their own right and/or be hydrolysable under in vivo conditions in the human body. Suitable pharmaceutically acceptable in vivo hydrolysable ester groups include those which break down readily in the human body to leave the parent acid or its salt.

The compounds of this invention may be in crystalline or non-crystalline form, and, if crystalline, may optionally be hydrated or solvated. Furthermore, some of the crystalline forms of the compounds of formula (IA) and (IB) may exist as polymorphs, which are included in the present invention.

The compounds according to the invention are suitably provided in substantially pure form, for example at least 50% pure, suitable at least 60% pure, advantageously at least 75% pure, preferably at least 85% pure, more preferably at least 95% pure, especially at least 98% pure, all percentages being calculated as weight/weight.

The compounds of formula (IA) and (IB) contain a carboxylic acid group and may therefore be in the form of a pharmaceutically acceptable salt. Compounds of the invention that contain a basic group such as an amino substituent may be in the form of a free base or an acid-addition salt. Compounds of the invention having both a basic and an acidic centre may be in the form of zwitterions, acid-addition salt of the basic centre or alkali metal salts (of the carboxylic acid group). Pharmaceutically acceptable salts are preferred.

Pharmaceutically acceptable acid-addition salts include those described by Berge, Bighley, and Monkhouse, J. Pharmi. Sci., 1977, 66, 1-19. Suitable addition salts are formed from acids which form non-toxic salts and examples are hydrochloride, hydrobromide, hydroiodide, sulphate, bisulphate, nitrate, phosphate, hydrogen phosphate, acetate, propionate, maleate, malate, fumarate, lactate, tartrate, citrate, formate, gluconate, succinate, salicylate, aspartate, stearate, palmate, itaconate, glycolate, piruvate, ascorbate, pamoate, succinate, bismethylenesalicylate, oxalate, oxaloacetate, trifluoroacetate, saccharate, benzoate, p-aminobenzoate, glutamate, methanesulphonate, ethanesulphonate, ethanedisulphonate, benzenesulphonate, p-toluenesulphonate, cyclohexylsulfamate, lactobionate and isethionate.

Pharmaceutically acceptable base salts Include those described by Berge, Bighley, and Monkhouse, J. Pharm. Sci., 1977, 66, 1-19. Suitable base salts include ammonium salts, alkali metal salts such as those of sodium and potassium, alkaline earth metal salts such as those of calcium and magnesium and salts with organic bases, including salts of primary, secondary and tertiary amines, such as isopropylamine, diethylamine, ethanolamine, trimethylamine, dicyclohexyl amine and N-methyl-D-glucamine. Representative salts include sodium and potassium salts.

Compounds of the present invention and pharmaceutically acceptable derivatives thereof may be readily prepared from available starting materials by adapting synthetic processes well known in the art. For example, the compounds of the invention and pharmaceutically acceptable derivatives thereof may be prepared by the processes described hereinafter, said processes constituting a further aspect of the invention. In the following description, the groups are as defined above for compounds of the invention unless otherwise stated.

Compounds of the present invention may be readily prepared from a pleuromutilin or a 19,20-dihydro-pleuromutilin derivative by adapting procedures well known in the art for forming ester groups. Suitable procedures are reviewed in, for example, I. O. Sutherland in Comprehensive Organic Chemistry, Vol. 2, ed. I. O. Sutherland, p. 869, Pergamon, 1979; and J. M. Brown, ibid., p. 779.

Accordingly, in a first aspect, the present invention provides a process for preparing a compound of formula (IA) or (IB) which comprises reacting a compound of formula (IIA) or (IIB): 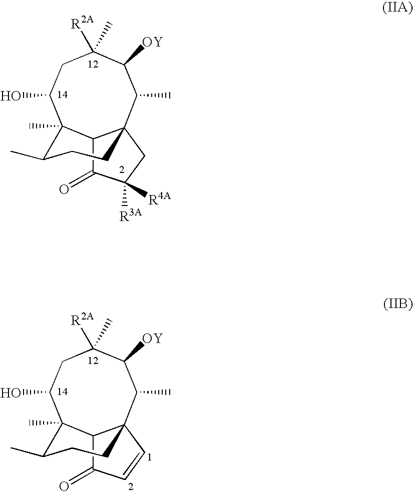
in which Y is hydrogen or a hydroxy protecting group, and R^{2A}, R^{3A} and R^{4A} are R², R³ and R⁴ as defined for formulae (IA) and (IB) or groups convertible R², R³ and R⁴,
with an active derivative of a carboxylic acid of formula (III):
R^{1A}SCH₂CO₂H (III)
where R^{1A} is R¹ as defined for formulae IA and IB or a group convertible to R¹, under ester forming conditions and, where required or desired,
converting Y to hydrogen,
converting an R^{2A}, R^{3A} and R^{4A} group to a R², R³ and R⁴ group, and/or
converting one R², R³ and R⁴ group to another R², R³ and R⁴ group.

Conventional methods for ester formation are described in the literature, for example in Comprehensive Organic Functional Group Transformations, Vol. 5, ed. C J Moody, p. 123-130, Elsevier Scientific, Oxford, 1995. The active derivative used as an acylating agent may be for example an acid chloride, acid bromide, a mixed anhydride, or an N-acyl-imidazole. The preferred agent is an acid chloride. General methods for forming such acylating agents are described in the chemical literature (see I O Sutherland, Comprehensive Organic Chemistry, Vol. 2, ed. I O Sutherland, pages 875-883 (Pergamon Press, Oxford, 1979), and references therein).

The ester-forming reaction can be carried out in the presence of an organic base, an inorganic base, or an acid. Organic bases include pyridine, 2,6-lutidine, triethylamine, and N,N-dimethylaniline. Inorganic bases include sodium hydride, lithium hydride, potassium carbonate, lithium hexamethyldisilazide, and sodium hexamethyldisilazide. Acids include p-toluenesulphonic acid, benzene sulphonic acid, and sulphuric acid. Optionally, when the reaction is carried out in the presence of a base, an acylation catalyst (G Hofle and W Steglich, Synthesis, 1972, 619) such as 4-dimethyamino-pyridine or 4-pyrrolidino-pyridine may also be added to the reaction mixture. Solvents for the ester forming reaction include Tetrahydrofuran, 1,4-dioxane, acetonitrile, N,N-dimethylformamide, diethyl ether, dichloromethane, and chloroform. A preferred solvent is Tetrahydrofuran.

Useful methods for acylating the 14-hydroxyl in the present invention include the use of the following: acid chloride in N,N-dimethylformamide at elevated temperature (e.g. 100° C. to 120° C.); acid chloride in the presence of an organic base (e.g. pyridine, 2,6-lutidine, 2,4,6-collidine, di-iso-propylethylamine) or an inorganic base (e.g. sodium or lithium hexamethyldisilazide); carboxylic acid in the presence of dicyclohexylcarbodiimide and an acylation catalyst (e.g. 4-dimethylamino-pyridine, 4-pyrrolidino-pyridine); a mutilin 14-chloroformate derivative plus carboxylic acid, tertiary base (e.g. triethylamine, di-iso-propyl-ethylamine), and an acylation catalyst (e.g. 4-dimethylamino-pyridine, 4-pyrrolidino-pyridine).

Those skilled in the art will appreciate that in the preparation of the compound of the invention or a solvate thereof it may be necessary and/or desirable to protect one or more sensitive groups in the molecule to prevent undesirable side reactions. Suitable hydroxy, carboxy and amino protecting groups are those well known in the art and which may be removed under conventional conditions and without disrupting the remainder of the molecule. A comprehensive discussion of the ways in which hydroxy, carboxy and amino groups may be protected and methods for cleaving the resulting protected derivatives is given in for example “Protective Groups in Organic Chemistry” (T. W. Greene, Wiley-Interscience, New York, 2nd edition, 1991) or “Protecting Group” by P. J. Kocienski (Georg Thieme Verlag 1994). Particularly suitable hydroxy protecting groups include, for example, triorganosilyl groups, for instance, trialkylsilyl such as trimethyl silyl or tert-butyidimethylsilyl; organocarbonyl and organooxycarbonyl groups, for instance, acetyl, allyloxycarbonyl, 4-methoxybenzyloxycarbonyl and 4-nitrobenzyloxycarbonyl; and alkyl ethers such as tertahydropyranyl. Particularly suitable carboxy protecting groups include alkyl and aryl groups, for instance methyl, ethyl and phenyl. Examples of suitable amino protecting groups include acyl type protecting groups (e.g. formyl, trifluoroacetyl, acetyl), aromatic urethane type protecting groups (e.g. benzyloxycarbonyl (Cbz) and substituted Cbz such as 4-methoxybenzyloxycarbonyl and 4-nitrobenzyloxycarbonyl), aliphatic urethane protecting groups (e.g. 9-fluorenylmethoxycarbonyl (Fmoc), alkoxycarbonyl such as t-butyloxycarbonyl (Boc) and isopropyloxycarbonyl, and cyclohexyloxycarbonyl) and alkyl type protecting groups (e.g. benzyl, trityl, chlorotrityl)

Conversions of an R^{2A}, R^{3A} or R^{4A} group to a R², R³ or R⁴ group typically arise when a protecting group is needed during the above coupling reaction or during the preparation of the reactants by the procedures described below. Interconversion of one R², R³ or R⁴ group to another typically arises when one compound of formula IA/B is used as the immediate precursor of another compound of formula IA/B or when it is easier to introduce a more complex or reactive substituent at the end of a synthetic sequence.

Preferably Y is a hydroxy protecting group such as an acyl group, for example so that —OY is trifluoroacetyl or dichloroacetyl. When the intended R³ is also hydroxyl, then R^{3A} is also preferably acyloxy, for example acetyl or dichloroacetyl. Hydroxyl groups at positions 11 and 2 (as groups OY and R^{3A}) may be protected using, for example, dichloroacetic anhydride and pyridine in Tetrahydrofuran or N-trifluoroacetyl-imidazole in Tetrahydrofuran at 0° C. After the reaction with the derivative of acid (III) is complete the protecting acyl groups may be removed to restore the hydroxyl groups by hydrolysis e.g. using NaOH in either MeOH or a Tetrahydrofuran/water solution.

It may also be necessary to protect substituent groups in the acid component (III) prior to reaction with the compound of formulae (IIA) or (IIB), for example protecting N atoms with alkoxycarbonyl, for example t-butoxycarbonyl.

R^{2A} is typically the R² group vinyl, and this may be converted to the alternative R² ethyl group by hydrogenating the vinyl group to form an ethyl group, typically by hydrogenation over a palladium catalyst (e.g. 10% palladium-on-carbon) in a solvent such as ethyl acetate, ethanol, dioxane, or Tetrahydrofuran.

R^{3A} is typically hydrogen or protected hydroxyl, such as acyloxy. After the coupling reaction, protecting acyl groups may be removed to restore the hydroxyl groups by hydrolysis e.g. using NaOH in MeOH.

A compound of formula (IA) may also be prepared from an epi-mutilin starting material. Accordingly, in another aspect, the present invention provides a process for preparing a compound of formula (IA) in which R³ and R⁴ are both hydrogen which comprises reacting an epi-mutilin compound of formula (IIC): 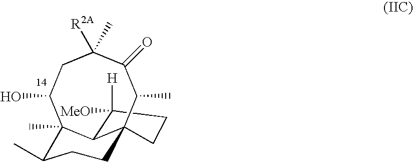
in which R^{2A} is R² as defined for formulae (IA) and (IB), or a group convertible to R²; with a compound of formula (III) as hereinbefore defined; to give a compound of formula (IV): 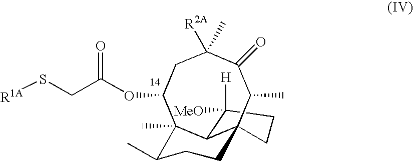
then treating the product with an acid and, where required or desired, converting an R^{1A} group to an R¹ group and an R^{2A} group to an R² group.

The acid treatment indicated above converts the epi-mutilin configuration of formula (IIC) to the usual mutilin nucleus of formula (IIA). Typically this conversion is carried out by treatment with conc. HCl or Lukas reagent (conc. HCl saturated with ZnCl₂) in dioxane.

As in formulae (IIA) and (IIB), R^{2A} is typically the R² group vinyl, and this may be converted to the alternative R² group by hydrogenating the vinyl group to form an ethyl group. Also it may again be necessary to protect substituent groups in the derivative of acid component (III) prior to reaction, for example protecting N atoms with, for example, t-butoxycarbonyl.

In cases where the intermediate of formula (IIA) and (IIB) (such as Y=acetyl) are used, a base-labile protecting group may conveniently be removed at the same time as the group Y is deprotected. In cases when the intermediate of formula (IIC) is used, an acid-labile protecting group may conveniently be removed at the same time as the acid treatment that converts the epi-mutilin configuration into the desired configuration of the compounds of the invention.

It should be appreciated that it may be necessary to interconvert one R¹, R², R³ or R⁴ group to another R¹, R², R³ or R⁴ group. This typically arises when one compound of formula (IA/B) is used as the immediate precursor of another compound of formula (IA/B) or when it is easier to introduce a more complex or reactive substituent at the end of a synthetic sequence. A substituent group in R¹ can be converted into another substituent group using one of the general methods for functional group transformation described in the literature (e.g. a carboxylic ester can be hydrolysed to a carboxylic acid with base; an acid can be converted into an amide; a tert-butoxycarbonylamino group can be converted into an amine by treatment with trifluoroacetic acid; an amino group can be alkylated or acylated), provided that the method chosen is compatible with other functional groups in the molecule (e.g. the ketone at C-3 in the pleuromutilin nucleus).

Functional group transformations are well known in the art and are described in, for instance, Comprehensive Organic Functional Group Transformations, eds. A. R. Katritzky, O. Meth-Cohn, and C. W. Rees (Elsevier Science Ltd., Oxford, 1995), Comprehensive Organic Chemistry, eds. D. Barton and W. D. Ollis (Pergamon Press, Oxford, 1979), and Comprehensive Organic Transformations, R. C. Larock (VCH Publishers Inc., New York, 1989).

Compounds of formulae (IIA) in which R^{3A} and R^{4A} are hydrogen, (IIB) and (IIC) may be readily prepared according to methods described in the literature, for example G. Schulz and H. Berner, Tetrahedron, 1984, 40, 905, and in WO 97/25309 and WO 98/05659 (SmithKline Beecham). Where necessary, and as hereinbefore described, saponification of the C-14 ester may be carried out at an appropriate stage.

Compounds of formula (IIA) in which R^{3A} is hydroxyl or fluoro may be prepared from pleuromutilin, via an intermediate 2-diazo compound, the preparation of which is described by G. Schulz and H. Berner in Tetrahedron, 1984, 40, 905. Where necessary, saponification of the C-14 ester group may be carried out at an appropriate stage using conventional techniques such as sodium hydroxide or sodium methoxide in methanol or aqueous Tetrahydrofuran solution.

The intermediate 2-diazo compound may be reacted with a carboxylic acid to give a 2-acyloxy-mutilin derivative. Suitably, reaction with dichloroacetic acid gives a 2-dichloroacetoxy-mutilin derivative, which can be deprotected as described above to provide the (2S)-2-hydroxy derivative, at an appropriate stage.

Compounds of formula (IIA) in which R^{3A} is fluoro may be obtained by reacting 2-diazo-mutilin with a source of hydrogen fluoride. Conveniently, the hydrogen fluoride source is an amine complex of hydrogen fluoride such as hydrogen fluoride-pyridine. The reaction may be carried out in an anhydrous solvent (e.g. diethyl ether, Tetrahydrofuran, 1,2-dimethoxyethane), at a temperature of −15° C. to 25° C. This reaction produces (2S)-2-fluoro derivatives. (2R)-2-Fluoro-mutilin derivatives may be prepared by treating the (2S)-isomer with a base (e.g. sodium hydroxide or potassium hydroxide in ethanol). This will usually produce a mixture of (2S) and (2R)-isomers that may be separated using conventional techniques such as chromatography and crystallisation.

Compounds of formula (IIB) are either 1,2-didehydro-mutilin or obtainable therefrom by manipulation of OY and R^{2A} as described above. 1,2-Didehydro-mutilins may be prepared using the method described by G Schulz and H Berner in Tetrahedron, 1984, 40, 905.

Compounds of formula (III) are available commercially.

The above described modifications to the mutilin nucleus may also be carried out after coupling of compounds of formula (IIA) and (IIC) where R^{3A} is hydrogen (i.e. based on mutilin and epi-mutilin) with the active derivative of acid (III).

In a preferred aspect, the present invention provides a method for preparing compounds of the invention which comprises reacting a compound of formula VA or VB 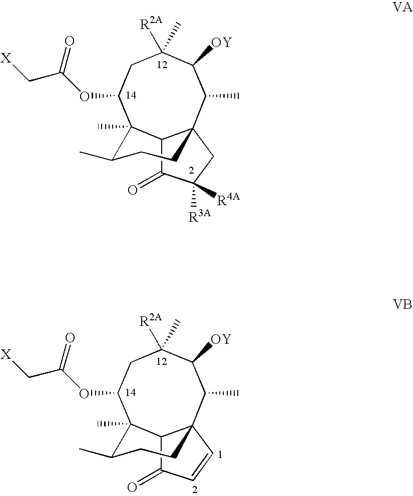
wherein X is a leaving group, Y is hydrogen or a hydroxy protecting group, and R^{2A}, R^{3A} and R^{4A} are R², R³ and R⁴ as defined for formulae IA and IB or groups convertible to R², R³ and R⁴,
with a compound of formula (VI):
R^{1A}SH (VI)
where R^{1A} is R¹ as defined for formulae (IA) and (IB) or a group convertible to R¹, by one of the procedures set out below and, where required or desired,
converting Y to hydrogen,
converting an R^{1A}, R^{2A}, R^{3A} or R^{4A} group to an R¹, R², R³ or R⁴ group, and/or
converting one R¹, R², R³ or R⁴ group to another R¹, R², R³ or R⁴ group.

X is a leaving group, for example a halogen or a reactive residue of a sulphonic acid, in particular a mesylate or tosylate group.

Y may be a hydroxy protecting group such as an acyl group, for example so that —OY is trifluoroacetyl or dichloroacetyl. Preferably Y is hydrogen. When the intended R³ is also hydroxyl then R^{3A} is preferably acyloxy, for example acetyl or dichloroacetyl.

It may also be necessary to protect substituent groups in the compound of formula (VI) prior to reaction with the compound (VA) or (VB), for example protecting N atoms with alkoxycarbonyl, for example t-butoxycarbonyl.

Suitable hydroxy, carboxy and amino protecting groups are those well known in the art and are discussed above.

R^{2A} is typically the R² group vinyl, and this may be converted to the alternative R² ethyl group by hydrogenating the vinyl group to form an ethyl group, typically by hydrogenation over a palladium catalyst (e.g. 10% palladium-on-carbon) in a solvent such as ethyl acetate, ethanol, dioxane, or Tetrahydrofuran.

R^{3A} is typically hydrogen or protected hydroxyl, such as acyloxy. After the coupling reaction, protecting acyl groups may be removed to restore the hydroxyl groups by hydrolysis e.g. using NaOH in MeOH.

Procedures for coupling the group XCH₂CO.O— with compound R^{1A}SH include, when R^{L} is a leaving group, such as 4-MeC₆H₄SO₂O, MeSO₂O, F₃CSO₂O, Br or Cl, reacting the thiol R^{1A}SH with the compound of formula (VA)/(VB) in the presence of an inorganic base in a appropriate solvent. Examples of suitable bases include sodium hydroxide, potassium hydroxide, sodium hydride, sodium methoxide, sodium ethoxide, sodium hexamethyldisilazide and lithium hexamethyldisilazide. Examples of suitable solvents include water, 2-propanol, ethanol, methanol, N,N-dimethylformamide, Tetrahydrofuran and 1,4-dioxane. The reaction is typically carried out at 0 to 40° C., preferably at room temperature.

Alternatively, the above reactions may be carried out using a compound of formula (VC): 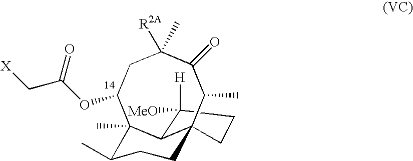
where X and R^{2A} are as defined for formulae VA and VB,
with the compound (VI) by the procedures set out above,
then treating the product with an acid and, where required or desired,
converting an R^{1A} or R^{2A} group to a R¹ or R² group, and/or
converting one R¹ or R² group to another R¹ or R² group.

As mentioned previously, the acid treatment indicated above converts the epi-mutilin configuration of formula (VC) to the usual mutilin nucleus of formula (VA). Typically this conversion is carried out by treatment with conc. HCl or Lukas reagent (conc. HCl saturated with ZnCl₂) in dioxane.

As in formulae (VA) and (VB), R^{2A} is typically the R² group vinyl, and this may be converted to the alternative R² group by hydrogenating the vinyl group to form an ethyl group. Also it may again be necessary to protect substituent groups in the compound (VII) prior to reaction, for example protecting N atoms with alkoxycarbonyl, for example t-butoxycarbonyl.

The compounds of formulae (VA), (VB) and (VC) may be prepared by reacting the corresponding compounds of formula (IIA), (IIB) and (IIC) by conventional methodology to introduce acyl groups substituted by a leaving group. For example, the preparation of a compound of formula (VB) from a compound of (IIB) is described in Example 57 of WO 99/21855 wherein the 14-chloroacetyl group is introduced by acylation using chloroacetylchloride in the presence of pyridine and N,N-dimethylaminopyridine.

Compounds of formulae (VA), (VB) and (VC) may be prepared from the corresponding 14-hydroxyacetyl analogues by the methods described by K Riedl in J. Antibiotics, 1976, 29, 132, and H Egger and H Reinshagen in J. Antibiotics, 1976, 29, 915 for pleuromutilin or 19,20-dihydro-pleuromutilin.

The compounds (VI) are commercially available or may be formed by conventional methodology from compounds that are commercially available compounds or described in the literature.

Where intermediates disclosed for the above processes are novel compounds, they also form part of this invention.

The compounds of the present invention may contain a chiral centre, and therefore the products of the above processes may comprise a mixture of diastereoisomers or a single diastereoisomer. A single diastereoisomer may be prepared by separating such a mixture of diastereoisomers which has been synthesised using a racemic starting material, or by synthesis using an optically pure starting material. Separation of diastereoisomers may be achieved by conventional techniques, e.g. by fractional crystallisation, chromatography or H.P.L.C.

The products of the processes of this invention may be In crystalline or non-crystalline form, and, if crystalline, may optionally be hydrated or solvated. When some of the compounds of this invention are allowed to crystallise or are recrystallised from organic solvents, solvent of crystallisation may be present in the crystalline product This invention includes within its scope such solvates. Similarly, some of the compounds of this invention may be crystallised or recrystallised from solvents containing water. In such cases water of hydration may be present in the crystalline product. This invention includes within its scope stoichiometric hydrates as well as compounds containing variable amounts of water that may be produced by processes such as lyophilisation.

The compounds obtained according to the processes of the invention are suitably worked up to a substantially pure form, for example at least 50% pure, suitable at least 60% pure, advantageously at least 75% pure, preferably at least 85% pure, more preferably at least 95% pure, especially at least 98% pure, all percentages being calculated as weight/weight. An impure or less pure form of a compound according to the invention may, for example, be used in the preparation of a more pure form of the same compound or of a related compound (for example a corresponding derivative) suitable for pharmaceutical use.

The present invention also includes pharmaceutically acceptable salts and derivatives of the compounds of the invention. Salt formation may be possible when one of the substituents carries an acidic or basic group. Salts may be prepared by salt exchange in conventional manner. Typically, a pharmaceutical acceptable salt may be readily prepared by using a desired acid or base as appropriate. The salt may precipitate from solution and be collected by filtration or may be recovered by evaporation of the solvent.

Acid-addition salts may be pharmaceutically acceptable or non-pharmaceutically acceptable. In the latter case, such salts may be useful for isolation and purification of the compound of the invention, or intermediates thereto, and will subsequently be converted into a pharmaceutically acceptable salt or the free base. Pharmaceutically acceptable acid-addition salts include those described by serge, Bighley, and Monkhouse, J. Pharm. Sci., 1977, 66, 1-19. Suitable salts are as described above.

The compounds of the present invention and their pharmaceutically acceptable salts or derivatives have antimicrobial properties and are therefore of use in therapy, in particular for treating microbial infections in animals, especially mammals, including humans, in particular humans and domesticated animals (including farm animals). The compounds may be used for the treatment of infections caused by, for example, Gram-positive and Gram-negative bacteria and mycoplasmas, including, for example, Staphylococcus aureus, Staphylococcus epidermidis, Enterococcus faecalis, Streptococcus pyogenes, Streptococcus agalactiae, Streptococcus pneumoniae, Haemophilus sp., Neisseria sp., Legionella sp., Chlamydia sp., Moraxella catarrhalis, Mycoplasma pneumoniae, and Mycoplasma gallisepticum.

Thus, the present invention provides a compound of the invention, or a pharmaceutically acceptable derivative thereof, for use in therapy.

The present invention also provides a method of treating microbial infections in animals, especially in humans and In domesticated mammals, which comprises administering a compound of the invention or a pharmaceutically acceptable salt or derivative or solvate thereof, or a composition according to the invention, to a patient in need thereof.

The invention further provides the use of a compound of the invention or a pharmaceutically acceptable salt or derivative or solvate thereof in the preparation of a medicament for use in the treatment of microbial infections.

Compounds of the present invention may be used to treat skin and soft tissue infections, for example secondarily infected dermotoses or traumatic lesions and impetigo, and acne, by topical application. Accordingly, in a further aspect the present invention provides the use of a compound of the invention or a pharmaceutically acceptable salt or derivative or solvate thereof in the preparation of a medicament adapted for topical administration for use in the treatment of skin and soft tissue infections and also in the treatment of acne in humans. The invention also provides the use of a compound of the invention; or a pharmaceutically acceptable derivative thereof, in the manufacture of a medicament for use in the treatment of a skin or soft tissue infection.

Compounds of the present invention may be also used for the elimination or reduction of nasal carriage of pathogenic bacteria such as S. aureus, H. influenzae, S. pneumonia and M. catarrhalis, in particular colonisation of the nasospharynx by such organisms, by the administration of a compound of the present invention thereto. Accordingly, in a further aspect, the present invention provides for the use of a compound of the invention or a pharmaceutically acceptable salt or derivative or solvate thereof in the manufacture of a medicament adapted for administration to the nasal cavity, for reducing or eliminating the nasal carriage of pathogenic organisms. Preferably, the medicament is adapted for focussed delivery to the nasopharynx, in particular the anterior nasopharynx.

Such reduction or elimination of nasal carriage is believed to be useful in prophylaxis of recurrent acute bacterial sinusitis or recurrent otitis media in humans, in particular in reducing the number of episodes experienced by a patient over a given period of time or increasing the time intervals between episodes. Accordingly, in a further aspect, the present invention provides for the use of a compound of the invention or a pharmaceutically acceptable salt or derivative or solvate thereof in the manufacture of a medicament adapted for administration to the nasal cavity, for prophylaxis of recurrent acute bacterial sinusitis or recurrent otitis media.

Compounds of the present invention are also useful in treating chronic sinusitis. Accordingly, in a further aspect, the present invention provides for the use of a compound of the invention or a pharmaceutically acceptable salt or derivative or solvate thereof in the manufacture of a medicament, for treating of chronic sinusitis.

It will be appreciated that reference to treatment includes acute treatment or prophylaxis as well as the alleviation of established symptoms.

Typically, a physician will determine the actual dosage which will be most suitable for an individual subject. The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

The compounds according to the invention may suitably be administered to the patient at a daily dosage of from 1.0 to 50 mg/kg of body weight. For an adult human (of approximately 70 kg body weight), from 50 to 3000 mg, for example about 1500 mg, of a compound according to the invention may be administered daily. Suitably, the dosage for adult humans is from 5 to 20 mg/kg per day. Higher or lower dosages may, however, be used in accordance with normal clinical practice.

To lessen the risk of encouraging the development of resistant organisms during prophylaxis of recurrent otitis media or recurrent acute bacterial sinusitis, it is preferred to administer the drug on an intermittent, rather than a continual, basis. In a suitable intermittent treatment regimen for prophylaxis of recurrent otitis media or recurrent sinusitis, drug substance is administered on a daily basis, for a small number of days, for instance from 2 to 10, suitably 3 to 8, more suitably about 5 days, the administration then being repeated after an interval, for instance, on a monthly basis over a period of months, for instance up to six months. Less preferably, the drug substance may be administered on a continuing, daily basis, over a prolonged period, for instance several months. Suitably, for prophylaxis of recurrent otitis media or recurrent sinusitis, drug substance is administered once or twice a day. Suitably, drug substance is administered during the winter months when bacterial infections such as recurrent otitis media and recurrent sinusitis tend to be more prevalent. The drug substance may be administered at a dosage of from 0.05 to 1.00 mg, typically about 0.1 to 0.2 mg, in each nostril, once or twice a day.

While it is possible that, for use in therapy, a compound of the present invention may be administered as the raw chemical, it is preferable to present the active ingredient as a pharmaceutical formulation e.g. when the agent is in admixture with a suitable pharmaceutical excipient, diluent or carrier selected with regard to the intended route of administration and standard pharmaceutical practice.

More specifically, the compounds and compositions according to the invention may be formulated for administration in any convenient way for use in human or veterinary medicine, by analogy with other antibiotics.

Accordingly, in one aspect, the present invention provides a pharmaceutical composition or formulation comprising at least one compound of the invention or a pharmaceutically acceptable derivative thereof in association with a pharmaceutically acceptable carrier and/or excipient. The carrier and/or excipient must be “acceptable” in the sense of being compatible with the other ingredients of the formulation and not deleterious to the recepient thereof.

In another aspect, the invention provides a pharmaceutical composition comprising a compound of the invention, or a pharmaceutically acceptable derivative thereof, and a pharmaceutically acceptable excipient, diluent or carrier.

In another aspect, the invention provides a pharmaceutical composition comprising, as active Ingredient, at least one compound of the invention or a pharmaceutically acceptable derivative thereof in association with a pharmaceutically acceptable carrier and/or excipient for use in therapy, and in particular, in the treatment of human or animal subjects suffering from a condition susceptible to amelioration by an antibacterial compound.

In another aspect, the invention provides a pharmaceutical composition comprising a therapeutically effective amount of the compounds of the present invention and a pharmaceutically acceptable excipient, diluent or carrier (including combinations thereof).

There is further provided by the present invention a process of preparing a pharmaceutical composition, which process comprises mixing at least one compound of the invention or a pharmaceutically acceptable derivative thereof, together with a pharmaceutically acceptable excipient, diluent and/or carrier.

The pharmaceutical compositions may be for human or animal usage in human and veterinary medicine and will typically comprise any one or more of a pharmaceutically acceptable excipient, diluent or carrier. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical diluent, excipient or carrier can be selected with regard to the intended route of administration and standard pharmaceutical practice. The pharmaceutical compositions may comprise as—or in addition to—the excipient, diluent or carrier, any suitable binder(s), lubricant(s), suspending agent(s), coating agent(s), solubilising agent(s), preservative(s), stabiliser(s), dye(s), flavouring agent(s) and antioxidant(s). Examples of preservatives include sodium benzoate, sorbic acid and esters of p-hydroxybenzoic acid.

For some embodiments, the agents of the present invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO 91/11172, WO 94/02518 and WO 98/55148.

The compounds of the invention may be milled using known milling procedures such as wet milling to obtain a particle size appropriate for tablet formation and for other formulation types. Finely divided (nanoparticulate) preparations of the compounds of the invention may be prepared by processes known in the art, for example see International Patent Application No. WO 02/00196 (SmithKline Beecham).

The routes for administration (delivery) include, but are not limited to, one or more of: oral (e.g. as a tablet, capsule, or as an ingestible solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, epidural, sublingual.

It is to be understood that not all of the compounds need be administered by the same route. Likewise, if the composition comprises more than one active component, then those components may be administered by different routes. For example, for some applications, preferably the agent is administered orally and for other applications, preferably the agent is administered topically.

There may be different composition/formulation requirements dependent on the different delivery systems. By way of example, the pharmaceutical composition of the present invention may be formulated to be delivered using a mini-pump or by a mucosal route, for example, as a nasal spray or aerosol for inhalation or ingestible solution, or parenterally in which the composition is formulated by an injectable form, for delivery, by, for example, an intravenous, intramuscular or subcutaneous route. Alternatively, the formulation may be designed to be delivered by both routes. In a preferred embodiment, the agents of the present invention are delivered topically. Hence, preferably the agent is in a form that is suitable for topical delivery.

The compounds and compositions according to the invention can be administered (e.g. orally or topically) in the form of tablets, capsules, powders, granules, lozenges, creams, syrups, sprays, ovules, elixirs, or liquid preparations, for example solutions or suspensions, which may be formulated for oral use or in sterile form for parenteral administration by injection or infusion and which may contain flavouring or colouring agents, for immediate-, delayed-, modified-, sustained-, pulsed-or controlled-release applications.

Tablets and capsules for oral administration may be in unit dosage form, and may contain conventional excipients including, for example, granulation binding agents, for example, syrup, acacia, gelatin, sorbitol, tragacanth, polyvinylpyrrolidone, hydroxypropylmethylcellulose (HPMC), hydroxypropylcellulose (HPC) and sucrose; fillers, for example lactose, sugar, maize-starch, calcium phosphate, sorbitol or glycine; tabletting lubricants, for example magnesium stearate, stearic acid, glyceryl behenate and talc, polyethylene glycol or silica; disintegrants, for example starch (preferably corn, potato or tapioca starch), sodium starch glycollate, croscarmellose sodium and certain complex silicates; pharmaceutically acceptable wetting agents, for example sodium lauryl sulphate; and other excipients such as microcrystalline cellulose, sodium citrate, calcium carbonate and dibasic calcium phosphate. The tablets may be coated according to methods well known in normal pharmaceutical practice.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

Oral liquid preparations may be in the form of, for example, aqueous or oily suspensions, solutions, emulsions, syrups or elixirs, or may be presented as a dry product for reconstitution with water or another suitable vehicle before use. Such liquid preparations may contain conventional additives, including, for example, suspending agents, for example sorbitol, methyl cellulose, glucose syrup, gelatin, hydroxyethyl cellulose, carboxymethyl cellulose, aluminium stearate gel or hydrogenated edible fats; emulsifying agents, for example lecithin, sorbitan monooleate or acacia; non-aqueous vehicles (which may include edible oils), for example almond oil, oily esters (for example glycerine), propylene glycol, or ethyl alcohol; preservatives, for example methyl or propyl p-hydroxybenzoate or sorbic acid; and, if desired, conventional flavouring and colour agents.

Where the agent is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

For buccal or sublingual administration the compositions may be administered in the form of tablets or lozenges which can be formulated in a conventional manner.

Compositions according to the invention intended for topical administration may, for example, be in the form of ointments, creams, lotions, solutions, dusting powders, eye ointments, eye drops, ear drops, nose drops, nasal sprays, impregnated dressings, and aerosols, and may contain appropriate conventional additives, including, for example, preservatives, solvents to assist drug penetration, and emollients in ointments and creams. Such topical formulations may also contain compatible conventional carriers, for example cream or ointment bases, ethanol or oleyl alcohol for lotions and aqueous bases for sprays. Such carriers may constitute from about 1% to about 98% by weight of the formulation; more usually they will constitute up to about 80% by weight of the formulation. For example, the agent of the present invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, it can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Compositions according to the invention intended for topical administration, in addition to the above, may also contain a steroidal anti-inflammatory agent; for example, betamethasone.

Compositions according to the invention may also be dermally or transdermally administered, for example, by the use of a skin patch.

Compositions according to the invention may be formulated as suppositories, which may contain conventional suppository bases, for example cocoa-butter or other glycerides. For example, the compound of the present invention can be administered in the form of a suppository or pessary, or it may be applied topically in the form of a gel, hydrogel, lotion, solution, cream, ointment or dusting powder.

Compositions according to the invention may also be administered by the pulmonary route, for example, by inhalation.

Compositions according to the invention may also be administered by the ocular route. For ophthalmic use, the compounds can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

Compositions according to the invention intended for parenteral administration may conveniently be in fluid unit dosage forms, which may be prepared utilizing the compound and a sterile vehicle, water being preferred. Examples of such administration include one or more of: intravenously, intraarterially, intraperitoneally, intrathecally, intraventricularly, intraurethrally, intrasternally, intracranially, intramuscularly or subcutaneously administering the agent; and/or by using infusion techniques. The compound, depending on the vehicle and concentration used, may be either suspended or dissolved in the vehicle. In preparing solutions, the compound may be dissolved in water for injection and filter-sterilised before being filled into a suitable vial or ampoule, which is then sealed. Advantageously, conventional additives including, for example, local anaesthetics, preservatives, and buffering agents can be dissolved in the vehicle. For example, the compound may be used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art. In order to enhance the stability of the solution, the composition may be frozen after being filled into the vial, and the water removed under vacuum; the resulting dry lyophilised powder may then be sealed in the vial and a accompanying vial of water for injection may be supplied to reconstitute the liquid prior to use. Parenteral suspensions may be prepared In substantially the same manner except that the compound is suspended in the vehicle instead of being dissolved and sterilisation cannot be accomplished by filtration. The compound may instead be sterilised by exposure to ethylene oxide before being suspended in the sterile vehicle. Advantageously, a surfactant or wetting agent is included in such suspensions in order to facilitate uniform distribution of the compound.

A compound or composition according to the invention is suitably administered to the patient in an antimicrobially effective amount.

A composition according to the invention may suitably contain from 0.001% by weight, preferably (for other than spray compositions) from 10 to 60% by weight, of a compound according to the invention (based on the total weight of the composition), depending on the method of administration.

When the compositions according to the invention are presented in unit dosage form, for instance as a tablet, each unit dose may suitably comprise from 25 to 1000 mg, preferable from 50 to 500 mg, of a compound according to the invention.

Representative compositions of the present invention include those adapted for intranasal administration, in particular, those that will reach into the nasopharynx. Such compositions are preferably adapted for focussed delivery to, and residence within, the nasopharynx. The term ‘focussed delivery’ is used to mean that the composition is delivered to the nasopharynx, rather than remaining within the nares. The term ‘residence’ within the nasopharynx is used to mean that the composition, once delivered to the nasopharynx, remains within the nasopharynx over a course of several hours, rather than being washed away more or less immediately. Preferred compositions include spray compositions and creams. Representative spray compositions include aqueous compositions, as well as oily compositions that contain amphiphilic agents so that the composition increases in viscosity when in contact with moisture. Creams may also be used, especially creams having a rheology that allows the cream to spread readily in the nasopharynx.

Preferred aqueous spray compositions include, in addition to water, further excipients including a tonicity modifier such as a salt, for instance sodium chloride; preservative, such as benzalkonium salt; a surfactant such as a non-ionic surfactant, for instance a polysorbate; and buffer, such as sodium dihydrogen phosphate; present in low levels, typically less than 1%. The pH of the composition may also be adjusted, for optimum stability of the drug substance during storage. For compounds of the present invention, a pH in the range 5 to 6, preferably about 5.3 to 5.8, typically about 5.5 is optimal.

Representative oily spray and cream compositions are described in WO 98/14189 (SmithKline Beecham). Representative aqueous sprays are described in International Application no PCT/GB98/03211 (SmithKline Beecham).

Suitably, the drug substance is present in compositions for nasal delivery in between 0.001 and 5%, preferably 0.005 and 3%, by weight of the composition. Suitable amounts include 0.5% and 1% by weight of the composition (for oily compositions and creams) and from 0.01 to 0.2% (aqueous compositions).

Spray compositions according to the present invention may be delivered to the nasal cavity by spray devices well known in the art for nasal sprays, for instance an air lift pump. Preferred devices include those that are metered to provide a unit volume of composition, preferably about 100 μl, and optionally adapted for nasal administration by addition of a modified nozzle.

When the compound of the present invention is administered intranasally or by inhalation and is conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134AT″″) or 1,1,1,2,3,3,3-heptafluoropropane (HFA 227EA), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate.

Capsules and cartridges (made, for example, from gelatin) for use in an inhaler or insufflator may be formulated to contain a powder mix of the compound and a suitable powder base such as lactose or starch.

The compounds of the invention may also be used in combination with other therapeutic agents. The invention thus provides, in a further aspect, a combination comprising a compound of the invention or a pharmaceutically acceptable derivative thereof together with a further therapeutic agent.

When a compound of the invention or a pharmaceutically acceptable derivative thereof is used in combination with a second therapeutic agent active against the same disease state the dose of each compound may differ from that when the compound is used alone. Appropriate doses will be readily appreciated by those skilled in the art. It will be appreciated that the amount of a compound of the invention required for use in treatment will vary with the nature of the condition being treated and the age and the condition of the patient and will be ultimately at the discretion of the attendant physician or veterinarian. The compounds of the present invention may be used in combination with other antibacterial drugs such as a penicillin, a cephalosporin, a sulfonamide or an erythromycin.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations by any convenient route.

When administration is sequential, either the compound of the invention or the second therapeutic agent may be administered first. When administration is simultaneous, the combination may be administered either in the same or different pharmaceutical composition.

When combined in the same formulation it will be appreciated that the two compounds must be stable and compatible with each other and the other components of the formulation. When formulated separately they may be provided in any convenient formulation, conveniently in such manner as are known for such compounds in the art.

The invention is illustrated by the following Examples.

### EXAMPLES

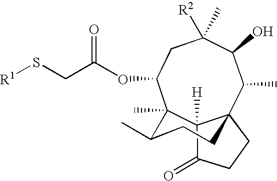

| | R¹ | R² |
|---|---|---|
| Example 1 | 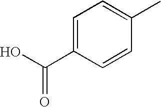 | vinyl |
| Examples 2 and 3 | 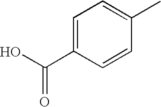 | ethyl |
| Example 4 | 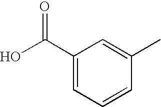 | vinyl |
| Example 5 | 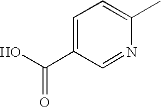 | vinyl |

### Example 1—(4-Carboxylato-phenylsulfanyl)-acetic acid mutilin 14-ester

A solution of methanesulfonyloxy-acetic acid mutilin 14-ester [65 g, 142 mmol; H. Egger and H. Reinshagen, J. Antibiotics, 1976, 29 (9), 915] in THF:water (2:1, 1200 ml) under an argon atmosphere was treated with 4-mercaptobenzoic acid (20 g, 130 mmol). 2M Aqueous sodium hydroxide (284 ml, 568 mmol) was added dropwise over 15 minutes and stirring continued at room temperature for a further 4 hours. The THF was removed by concentration under reduced pressure, and the resultant aqueous solution diluted with water to make a total volume of 800 ml. This was washed with ethyl acetate (2×400 ml), and the resultant aqueous phase adjusted to pH 4.5 with 5M HCl. The resultant white precipitate was collected by filtration, washed with water and dried in vacuo at 40° C., to give the title compound as a white solid (55 g, 80%). Crystallisation from acetone:water (4:5, 2.4 litres) afforded white crystals, m.p. 218.3° C.; ¹H NMR (d₆-DMSO) inter alia 0.59 (3H, d, J=7 Hz), 0.81 (3H, d, J=7 Hz), 0.98 (3H,s), 1.32 (3H,s), 2.04-2.18, 3H,m), 2.37 (1H,s), 3.38, (1H, t, J=6 Hz), 3.93 and 3.98 (2H, ABq, J=16 Hz), 4.48 (1H, d, J=6 Hz), 4.94-4.97 (2H, m), 5.51 (1H, d, J=8 Hz), 6.03 (1H, dd, J 17, 11 Hz), 7.42 (2H,d, J=9 Hz), 7.82 (2H, d, J=9 Hz), 12.89 (1H, s); MS (ES⁻) m/z 513 (M-H)⁻.

### Example 2—(4-Carboxylato-phenylsulfanyl)-acetic acid 19,20-dihydro-mutilin 14-ester

A solution of (4-carboxylatophenyl-sulfanyl)-acetic acid mutilin 14-ester (2.0 g, 3.99 mmol) in ethanol (100 ml) was hydrogenated over 10% Pd/C (4.0 g) at 50 psi for 48 hours. The catalyst was removed by filtration though Kieselguhr and the solvent evaporated. The crude product was crystallised from methanol to gave the title compound as a white solid (0.82 g, 38%); MS (ES⁻) m/z 515 (M-H)⁻.

### Example 3—(4-Carboxylato-phenylsulfanyl)-acetic acid 19,20-dihydro-mutilin 14-ester

19,20-Dihydropleuromutilin (88.5 g, 0.212 mol) was dissolved in dichloromethane (700 ml) with ice-bath cooling, and treated with triethylamine (32.4 ml, 0.254 mol) followed by methane sulfonyl chloride (18.03 ml, 0.233 mol). The reaction mixture was stirred at room temperature overnight, then washed with water and brine, dried (Na₂SO₄) and evaporated to give methanesulfonyloxy-acetic acid 19,20-dihydro-mutilin 14-ester as a foam.

Methanesulfonyloxy-acetic acid 19,20-dihydro-mutilin 14-ester (8.3 g, 18.3 mmol) was reacted with 4-mercaptobenzoic acid (2.2 g, 15.4 mmol) under the conditions described in Example 1. After acidification of the aqueous phase, the product was extracted into ethyl acetate and the combined organic extracts dried (Na₂SO₄) and evaporated to give the title compound as a solid (3.0 g), which was recrystallised from methanol to give material identical to that obtained in Example 2.

### Example 4—(3-Carboxylato-phenylsulfanyl)-acetic acid mutilin 14-ester

Methanesulfonyloxy-acetic acid mutilin 14-ester (0.5 g, 1.1 mmol) was reacted with 3-mercaptobenzoic acid (136 mg, 0.88 mmol) in THF (14 ml) and water (7 ml) under the conditions described in Example 1. After acidification of the aqueous phase, the product was extracted into ethyl acetate and the combined organic extracts dried (Na₂SO₄) and evaporated to give the title compound as a white solid (370 mg, 65%); MS (ES⁻) m/z 513 (M-H)⁻; MS (ES⁺) m/z 532 (MNH₄)⁺.

### Example 5—(5-Carboxylato-pyridin-2-yl-sulfanyl)-acetic acid mutilin 14-ester

Using the process described in Example 4, the title compound was prepared as a yellow solid (32% yield); MS (ES⁺), m/z 516 (MH⁺).

### Example 6—(4-Carboxylato-phenylsulfanyl)-acetic acid mutilin 14-ester. Sodium Salt

(4-Carboxylato-phenylsulfanyl)-acetic acid mutilin 14-ester (Example 1) (5.14 g, 10.0 mmol) in methanol (100 ml) was treated with aqueous sodium hydroxide solution (2M, 5.0 ml). After stirring for 30 mins, the solution was evaporated, and the solid dissolved in water (120 ml). The pH was checked (pH 7-8), and the solution filtered through Kieselguhr to remove any fine suspension. The resultant solution was lyophilised to afford the title compound as a fluffy white solid (5.7 g). NMR showed a shift in the aromatic protons compared to the corresponding free acid; ¹H NMR (d₆-DMSO) inter alia 7.20 (2H,d, J=8.4 Hz), 7.75 (2H, d, J=8.4 Hz), 12.89 (1H, s); MS (ES⁻) m/z 513 (M-H)⁻.

### Example 7—(4-Carboxylato-phenylsulfanyl)-acetic acid mutilin 14-ester. Potassium Salt

(4-Carboxylato-phenylsulfanyl)-acetic acid mutilin 14-ester (Example 1) (2.57 g, 5.0 mmol) in methanol (50 ml) was treated with aqueous potassium hydroxide solution (1M, 5.0 ml), following the procedure of Example 6. The product was lyophilised to afford the title compound as a fluffy white solid (2.8 g). NMR showed a shift in the aromatic protons compared to the corresponding free acid; ¹H NMR (d₆-DMSO) inter alia 7.19 (2H,d, J=8.4 Hz), 7.73 (2H, d, J=8.4 Hz), 12.89 (1H, s); MS (ES⁻) m/z 513 (M-H)⁻.

Biological Data

The compounds prepared in Examples 1-5 were tested for antibacterial activity using conventional methodology. Against 30 clinical isolates of Staphylococcus aureus the compounds gave MIC90 values (concentrations at which growth is inhibited for 90% of the strains) of 0.12 to 0.25 micrograms per millilitre. Against a panel of 10 isolates of Streptococcus pyogenes the compounds also showed strong activity with MICs of ≦0.06 to 0.25 micrograms per millilitre. The activity of Example 1 as compared to (2-carboxylato-phenylsulfanyl)-acetic acid mutilin 14-ester, expressed as MICs in micrograms per millillitre, is summarised in the table below.

| | Compound | |
|---|---|---|
| | | (2-carboxylato- |
| | | phenylsulfanyl)- |
| Organism | Example 1 | acetic acid mutilin 14-ester |
| Staphylococccus aureus Oxford | 0.125 | 0.5 |
| Streptococcus pneumoniae 1629 | 0.5 | 8 |

We claim:

1. A compound of formula (IA) or (IB): 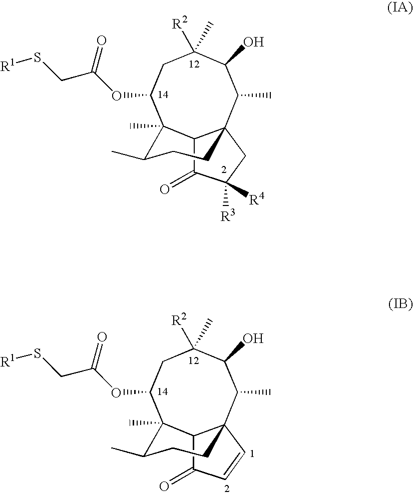
wherein:
R¹ is phenyl or a five- or six-membered heteroaryl ring, wherein R¹ is substituted by a carboxylic acid group that is attached at a position which is not adjacent to the point of attachment of the sulphur, and wherein R¹ is optionally further substituted by up to four groups independently selected from halogen, (C₁₋₆)alkyl, aryl, aryl(C₁₋₆)alkyl, (C₁₋₆)alkoxy, (C₁₋₆)alkoxy(C₁₋₆)alkyl, halo(C₁₋₆)alkyl, aryl(C₁₋₆)alkoxy, hydroxy, nitro, cyano, azido, amino, mono- and di-N-(C₁₋₆)alkylamino, acylamino, arylcarbonylamino, acyloxy, carbamoyl, mono- and di-N-(C₁₋₆)alkylcarbamoyl, (C₁₋₆)alkoxycarbonyl, aryloxycarbonyl, ureido, guanidino, (C₁₋₆)alkylguanidino, amidino, (C₁₋₆)alkylamidino, sulphonylamino, aminosulphonyl, (C₁₋₆)alkylthio, (C₁₋₆)alkylsulphinyl, (C₁₋₆)alkylsulphonyl, heterocyclyl, heteroaryl, heterocyclyl(C₁₋₆)alkyl and heteroaryl(C₁₋₆)alkyl, or two adjacent ring carbon atoms may be linked by a (C₃₋₅)alkylene chain, to form a carbocyclic ring;
R² is vinyl or ethyl; and
R³ is hydrogen, hydroxy or fluorine and R⁴ is hydrogen,
or R³ is hydrogen and R⁴ is fluorine;
or a pharmaceutically acceptable derivative thereof

2. A compound according to claim 1 wherein R¹ is a five- or six-membered aryl ring or a five- or six-membered heteroaryl ring containing up to three heteroatoms independently selected from nitrogen, sulphur or oxygen, substituted by a carboxylic acid group.

3. A compound according to claim 1 or 2 wherein R¹ is a six-membered aryl ring or a six-membered heteroaryl ring containing one or two nitrogen atoms, substituted by a carboxylic acid group.

4. A compound according to claim 1 wherein R¹ is phenyl or pyridyl, substituted by a carboxylic acid group.

5. A compound according to claim 1 selected from:
(4-carboxylato-phenylsulfanyl)-acetic acid mutilin 14-ester;
(4-carboxylato-phenylsulfanyl)-acetic acid 19,20-dihydro-mutilin 14-ester;
(3-carboxylato-phenylsulfanyl)-acetic acid mutilin 14-ester; and
(5-carboxylato-pyridin-2-yl-sulfanyl)-acetic acid mutilin 14-ester;
or a pharmaceutically acceptable derivative thereof.

6. A pharmaceutical composition comprising a compound as claimed in claim 1, or a pharmaceutically acceptable derivative thereof, and a pharmaceutically acceptable excipient, diluent or carrier.

7. A compound as claimed in claim 1, or a pharmaceutically acceptable derivative thereof, for use in therapy.

8. (canceled)

9. (canceled)

10. A method of treating microbial infections in animals, which comprises administering a compound according to claim 1, or a pharmaceutically acceptable derivative thereof, or a composition according to the invention, to a patient in need thereof.

11. A method of treatment of skin and soft tissue infections in humans, which comprises topically administering a compound according to claim 1, or a pharmaceutically acceptable derivative thereof, or a composition according to the invention, to a patient in need thereof.

12. A process for preparing a compound of formula (IA) or (IB) as claimed in claim 1 which process comprises:
(a) reacting a compound of formula (IIA) or (IIB): 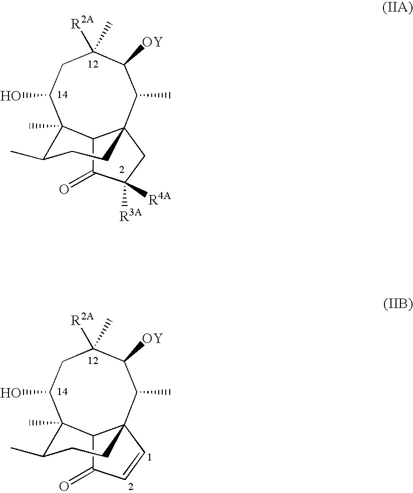
in which Y is hydrogen or a hydroxy protecting group, and R^{2A}, R^{3A} and R^{4A} are R², R³ and R⁴ as defined in claim 1 or groups convertible R², R³ and R⁴, with an active derivative of a carboxylic acid of formula (III):
R^{1A}SCH₂CO₂H (III)
where R^{1A} is R¹ as defined in claim 1 or a group convertible to R¹, under ester forming conditions and, where required or desired,
converting Y to hydrogen,
converting an R^{2A}, R^{3A} and R^{4A} group to a R², R³ and R⁴ group, and/or
converting one R², R³ and R⁴ group to another R², R³ and R⁴ group;
(b) for a compound of formula (IA) in which R³ and R⁴ are both hydrogen, reacting an epi-mutilin compound of formula (IIC): 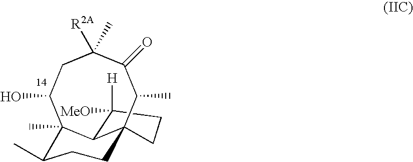
in which R^{2A} is R² as defined in claim 1, or a group convertible to R²;
with a compound of formula (III) as hereinbefore defined;
to give a compound of formula (IV): 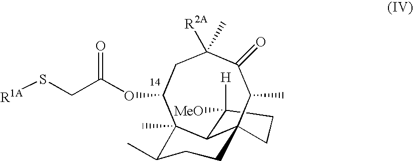
then treating the product with an acid and, where required or desired, converting an R^{1A} group to an R¹ group and an R^{2A} group to an R² group;
(c) reacting a compound of formula VA or VB 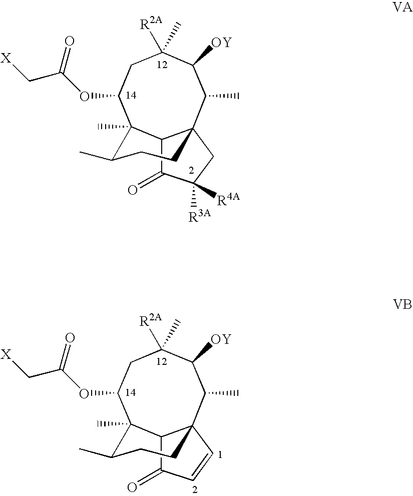
wherein X is a leaving group, Y is hydrogen or a hydroxy protecting group, and R^{2A}, R^{3A} and R^{4A} are R², R³ and R⁴ as defined in claim 1 or groups convertible to R², R³ and R⁴,
with a compound of formula (VI):
R^{1A}SH (VI)
where R^{1A} is R¹ as defined in claim 1 or a group convertible to R¹ and, where required or desired,
converting Y to hydrogen,
converting an R^{1A}, R^{2A}, R^{3A} or R^{4A} group to an R¹, R², R³ or R⁴ group, and/or
converting one R¹, R², R³ or R⁴ group to another R¹, R², R³ or R⁴ group; or
(d) reacting a compound of formula (VC): 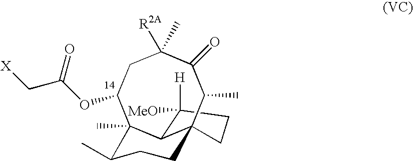
where X and R^{2A} are as defined for formulae VA and VB,
with the compound (VI),
then treating the product with an acid and, where required or desired,
converting an R^{1A} or R^{2A} group to a R¹ or R² group, and/or
converting one R¹ or R² group to another R¹ or R² group.
